SEQUENCE LISTING (2 sequences)

SEQ ID NO: 1 - DNA, 3470 bp, Homo sapiens
gagctccaccgcggtggcggccgctctgagaactagtggatccactagtaacggccgccagtgtgctggaattcgcccttgcaaatatatatatatatatatattttttttttttttttagagataagagtcttgctctgtcgcctaggctggagtgcagtggcacaatctctgctcactgcaacctccgcctccagggttcaagtgattctgctgcctcagcctcccaggtgggattacaggtgcctgccaccacgcctggctaatttttttgtctttttagtaaagatgaggtttcaccatgttgggcaggctggtttcaattgctgacctcaagtgagccaccccgcctcagcctcccaaaatgctaggattacaggcatgagccaccgcacccagccaagtttgtacatatatttttgactacacttcttaactattcttaggataaattactagaagtgaaaattctggggtgaagagcttgaggcctttacacacacacacacacacacacacacacacacaaataggctggatgcagtggctcacacctgtaatctcagcagtttgggaggctgaggaaggaggatcacttgagtccaggaggttgagaatagcctgaacaacatagcaagatcttgtctctacaaaaaattaaaaaaaaattagctggccatggcagcatgtgcctgtagtaccagctactcggaaggctgaggtaggaggatcgcttgagcccaggaggttgattgaagctgcagtgagctgtgattacaccactgcactccagcctgggcaacagagctagactctgtctctaaaaaaagcacaaaataatatttaaaaagcaccaggtatgcctgtacttgagttgtctttgttgatggctacaaatgaggacagctctggctgaagggcgcttccatttccatgggctgaaggagggacattttgcaaagtgtgttttcaggaagacacagagttttacctcctacacttgtttgatctgtattaatgtttgcttatttatttatttaattttttttttgagacagagtctcactctgtcacctgggctggagtgcagtggcattattgaggctcattgcagtctcagactcctgagctcaaacaatcctcctgcctcagcctctggagtagctaggactacaggcatgtgccaccatgcctggctaattttttaaatgtatttttttgtagagtcggggtctccctatgttgcccaggctggagtgcagtggtgtgatcctagctcactgcagcctggacctcgggctcaagtaattctcacacctcagcctgtccagtagcaggggctacaggcgcgcaccaccatgcccagctaattaaaaatatttttttgtagagacagggtctctctatgttgcccaggctggtttcaaactcccaggctcaagcaatcctcctgccttggcctcccaaagtgctggcattacaggcgtgagccactgcgcctggcccgtattaatgtttagaacacgaattccaggaggcaggctaagtctattcagcttgttcatatgcttgggccaacccaagaaacaagtgggtgacaaatggcaccttttggatagtggtattgactttgaaagtttgggtcaggaagctggggaggaagggtgggcaggctgtgggcagtcctgggcggaagaccaggcagggctatgtgctcactgagcctccgccctcttcctttgaatctctgatagacttctgcctcctacttctccttttctgcccttctttgctttggtggcttccttgtggttcctcagtggtgcctgcaacccctggttcacctccttccaggttctggctccttccagccaagggcgaattctgcagatatcgaagttggtcgtgaggcactgggcaggtaagtatcaaggttacaagacaggtttaaggagaccaatagaaactgggcttgtagagacagagaagactcttgcgtttctgataggcacctattggtcttactgacatccactttgcctttctctccacaggtgtccactcccagttcaattacagctcttaaggctagagtacttaatacgactcactataaagcttgccgccaccatggagacaaattcctctctccccacgaacatctctggagggacacctgctgtatctgctggctatctcttcctggatatcatcacttatctggtatttgcagtcacctttgtcctcggggtcctgggcaacgggcttgtgatatgggtggctggattccggatgacacacacagtcaccaccatcagttacctgaacctggccgtggctgacttctgtttcacctccactttgccattcttcatggtcaggaaggccatgggaggacattggcctttcggctggttcctgtgcaaattcgtctttaccatagtggacatcaacttgttcggaagtgtcttcctgatcgccctcattgctctggaccgctgtgtttgcgtcctgcatccagtctggacccagaaccaccgcaccgtgagcctggccaagaaggtgatcattgggccctgggtgatggctctgctcctcacattgccagttatcattcgtgtgactacagtacctggtaaaacggggacagtagcctgcacttttaacttttcgccctggaccaacgaccctaaagagaggataaatgtggccgttgccatgttgacggtgagaggcatcatccggttcatcattggcttcagcgcacccatgtccatcgttgctgtcagttatgggcttattgccaccaagatccacaagcaaggcttgattaagtccagtcgtcccttacgggtcctctcctttgtcgcagcagccttttttctctgctggtccccatatcaggtggtggcccttatagccacagtcagaatccgtgagttattgcaaggcatgtacaaagaaattggtattgcagtggatgtgacaagtgccctggccttcttcaacagctgcctcaaccccatgctctatgtcttcatgggccaggacttccgggagaggctgatccacgcccttcccgccagtctggagagggccctgaccgaggactcaacccaaaccagtgacacagctaccaattctactttaccttctgcagaggtggagttacaggcaaagtgaaagcttatcgataccgtcgacctcgaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaggtacc

SEQ ID NO: 2 - DNA, 1053 bp, Homo sapiens
atggagacaaattcctctctccccacgaacatctctggagggacacctgctgtatctgctggctatctcttcctggatatcatcacttatctggtatttgcagtcacctttgtcctcggggtcctgggcaacgggcttgtgatctgggtggctggattccggatgacacacacagtcaccaccatcagttacctgaacctggccgtggctgacttctgtttcacctccactttgccattcttcatggtcaggaaggccatgggaggacattggcctttcggctggttcctgtgcaaattcgtctttaccatagtggacatcaacttgttcggaagtgtcttcctgatcgccctcattgctctggaccgctgtgtttgcgtcctgcatccagtctggacccagaaccaccgcaccgtgagcctggccaagaaggtgatcattgggccctgggtgatggctctgctcctcacattgccagttatcattcgtgtgactacagtacctggtaaaacggggacagtagcctgcacttttaacttttcgccctggaccaacgaccctaaagagaggataaatgtggccgttgccatgttgacggtgagaggcatcatccggttcatcattggcttcagcgcacccatgtccatcgttgctgtcagttatgggcttattgccaccaagatccacaagcaaggcttgattaagtccagtcgtcccttacgggtcctctcctttgtcgcagcagccttttttctctgctggtccccatatcaggtggtggcccttatagccacagtcagaatccgtgagttattgcaaggcatgtacaaagaaattggtattgcagtggatgtgacaagtgccctggccttcttcaacagctgcctcaaccccatgctctatgtcttcatgggccaggacttccgggagaggctgatccacgcccttcccgccagtctggagagggccctgaccgaggactcaacccaaaccagtgacacagctaccaattctactttaccttctgcagaggtggagttacaggcaaagtga

(19) United States
(12) Patent Application Publication
(10) Pub. No.: US 2012/0216303 A1
(43) Pub. Date: Aug. 23, 2012

(54) NOVEL THERAPEUTIC USES OF HUMAN FORMYL PEPTIDE RECEPTOR ANTAGONISTS

(75) Inventor: John D. BENSON, West Roxbury, MA (US)

(73) Assignee: NIKAN PHARMACEUTICALS, LLC, West Roxbury, MA (US)

(21) Appl. No.: 12/723,163

(22) Filed: Mar. 12, 2010

Related U.S. Application Data
(63) Continuation of application No. 10/794,925, filed on Mar. 5, 2004, now abandoned.
(60) Provisional application No. 60/452,892, filed on Mar. 7, 2003.

(51) Int. Cl. A01K 67/027; C12Q 1/02; A61P 1/00; A61K 38/13; A61P 11/00

(52) U.S. Cl. 800/9; 514/20.5; 435/29

(57) ABSTRACT

The invention features a transgenic mouse that expresses human formyl peptide receptor and methods for producing this mouse. The invention also features methods for the measurement of an inflammatory response, particularly that associated with cystic fibrosis. The methods of the invention also feature methods for determining whether a compound inhibits or prevents the recruitment of neutrophils.

### CROSS REFERENCE TO RELATED APPLICATIONS

This application is a continuation of U.S. application Ser. No. 10/794,925, filed Mar. 5, 2004, which claims the benefit of U.S. Provisional Application No. 60/452,892, filed Mar. 7, 2003.

### BACKGROUND OF THE INVENTION

Neutrophils play a crucial role in defense against infection by being among the first cells that migrate to sites of infection to eliminate foreign bodies from those sites. They are abundant in the blood, but absent from normal tissue. Upon chemotactic recruitment to a site of infection and activation through various signaling molecule gradients that stimulate cellular receptors at nanomolar concentrations (i.e., chemoattractants, interleukins, and chemokines), neutrophils initiate a cascading cellular and physiological response, ultimately resulting in the release of superoxide and elastase, the release of other factors that further amplify the immune response, recruitment of monocytes, and phagocytosis of the antigenic body. While these events represent important physiological components of innate immune response, several pathological conditions may be associated with inappropriate or exaggerated activation of neutrophils and thereby cause excessive tissue damage.

Leukocyte recruitment from the blood vascular endothelium, endothelial transmigration, and recruitment to surrounding tissues is a complicated process that involves the integration of multiple signaling gradients, the order and concentration of which may define the intensity, duration, and outcome of the response (Foxman et al., J. Cell Biol. 147: 577-587 (1999)). A number of small molecules are known to be important mediators of neutrophil responses, including pro-inflammatory cytokines such as tumor necrosis factor alpha (TNF-α), interleukin-8 (IL-8), IL-1, and leukotriene-B4 (LTB4), as well as end target-derived chemoattractants formyl-methionyl peptides and C5a. Little is known about the relative physiological roles of various chemoattractants in the recruitment and activation of neutrophils at sites of infection or inflammation. It is unclear whether these respective factors serve redundant or separable functions in the initiation, amplification, perpetuation, and orchestration of localized immune response.

As early as 1954, it was observed that extracts of tissues infected with viable bacteria contain neutrophil and macrophage attractants (Harris, Physiol. Rev. 34:529-562 (1954)). These factors were later discovered to be N-formylmethionyl peptides which are present in filtrates of both gram-positive and gram-negative bacteria (Schiffmann et al., Proc. Natl. Acad. Sci. USA 72:1059-1062 (1975); Schiffmann et al., J. Immunol. 114:1831-18 (1975)). Immune response to bacterial pathogens results in the release of degraded peptides containing formylated methionine, that serve as highly potent chemoattractants for leukocyte and macrophage migration and infiltration. The receptor for these peptides has been cloned (Murphy et al., FEBS Lett. 261:353-357 (1990); Perez et al., Biochemistry 31:11595-11599 (1992)) and identified as a seven transmembrane G protein coupled receptor (GPCR). The cellular response mediated by binding of formylated peptide antagonists to the formyl peptide receptor (FPR) includes cellular polarization and transmigration, generation of superoxide O₂⁻ radicals through respiratory burst oxidase, degranulation and release of a variety of various degradative enzymes, as well as phagocytosis.

Experiments in some mouse models have suggested the desirability of eliminating one immune response component instead of another. For example, response to bacterial infection has been studied in FPR knockout mice (Gao et al., J. Exp. Med. 169:657-662 (1999)). The FPR −/− mice exhibit increased mortality following a high-dose intravenous Listeria monocytogenes challenge; however, the outcome of P. aeuroginosa lung infection in these mice has not been tested directly. It would appear that the FPR response is relatively specific because no developmental defects were observed and these mice were not susceptible to spontaneous infections. In contrast, loss of more systemically acting immunomodulatory signaling molecules (e.g., IL-8) results in many defects, including both neutrophil and T cell migration (reviewed in Ben-Baruch et al., J. Biol. Chem. 270:11703-11706 (1995)). In addition, transgenic mice having a C5a receptor (C5aR) deletion are unable to clear intrapulmonary instilled Pseudomonas aeruginosa despite an increase in neutrophil influx (Hopken et al., Nature 383: 86-89 (1996)). Thus, in contrast to other components of chemotactic signaling in the immune response, C5a has a non-redundant function and is essential for mucosal host defense in the lung.

Categorically, GCPRs have been the most successful target proteins for therapeutic intervention in disease processes: an estimated 30% of clinically prescribed drugs are GPCR agonists or antagonists (Stadel et al., Trends Pharmacol. Sci. 18:430-437 (1997)). The formyl peptide receptor (FPR) is a GPCR. Thus, although the potential of the FPR as a potential target for anti-inflammatory therapy is largely unexplored, FPR antagonists may be of considerable interest for use in treatment of inflammation-related disorders.

In addition to formylated peptide ligands, a number of FPR agonists and antagonists have been discovered. Antagonists include derivatized short Leu-Phe-peptides, as well as the cyclic cyclosporin H (CsH) peptide. CsH is a stereoisomer of cyclosporin A (CsA) that contains D- instead of L-valine at the eleventh peptide residue. In contrast to CsA which is used clinically as an immunosuppressant, CsH is not considered to be immunosuppressive and does not bind cyclophillin. The only known biological activity of CsH is as a potent FPR antagonist.

The Leu-Asp-Leu-Leu-Phe-Leu (LDLLFL) peptide represents another class of FPR ligand. LDLLFL is a fragment of CKS-17; a protein derived from a conserved region of retroviral transmembrane proteins that has been shown to suppress several different components of immune response. LDLLFL is an antagonist of the FPR that competes for formyl-Met-Leu-Phe (fMLP) binding (Oostendorp et al., J. Immunol. 149: 1010-1015 (1992); Oostendorp et al., J. Leukocyte Biol. 51: 282-288 (1992); Oostendorp et al., Eur. J. Immunol. 22: 1505-1511 (1995)).

The annexin I protein is another class of FPR ligand. The anti-inflammatory activity of annexin I has been observed in many systems, although the mechanism of these effects is not well understood. More recently, peptides derived from N-terminal proteolytic fragments of annexin I have been found to be FPR ligands. The 26 amino acid N-terminal peptide that is produced by the cleavage of full length annexin I by neutrophil elastase is an FPR agonist, although it acts at much higher concentrations than fMLP (K_{d}=740 μm and 0.02 μm, respectively). Tests of the effects of this agonist on cells expression FPR suggest that, although it is an agonist, this peptide may attenuate immune response by effectively inducing receptor desensitization.

This annexin I fragment is abundant in BAL samples from the lungs of cystic fibrosis (CF) patients and smokers, but is not found in normal lung samples. These fragments may be secondary to the effects of neutrophil infiltration in these lungs and could act to attenuate inflammation in these tissues. Alternatively, these fragments may contribute to the etiology or perpetuation of inflammation. Hyperstimulation of neutrophil activation through FPR activation may suggest FPR antagonism as an alternate strategy for attenuation of chronic lung inflammation.

Cystic fibrosis is the most common hereditary disorder in Caucasians. Airway inflammation is a major factor in its pathogenesis, with chronic Pseudomonas aeuruginosa lung infections the greatest cause of morbidity and mortality in individuals with CF. Such infections in CF patients elicit massive neutrophil infiltration that is unaccompanied by bacterial clearance. Antibiotic therapy typically has limited efficacy that diminishes over time. CF patients eventually die of progressive lung deterioration.

Treatment of CF at present is symptomatic, consisting of antibiotic therapy to combat bacterial infections, physiotherapy to remove the viscous mucus from the airways, the administration of pancreatic enzymes to compensate for the loss of pancreatic function (found in at least 85% of patients), the administration of mucolytic agents, and dietary regulations to optimize energy intake. Short-term, high-dose ibuprofen or other anti-inflammatory agents can be useful in addressing the profound and protracted inflammation characteristic of the CF lung, but toxicity to the liver and other organ prevent their long-term use. The efficacy of anti-inflammatory drugs in CF patients does however, validate the desirability and utility of addressing inflammation as a key mode of effective therapy. Although the basic defect and the gene responsible for the disease have been known for the past 10 years, a successful and suitable treatment for the basic defect in ion transport is not yet available.

CF airway inflammation is characterized by unusually dense neutrophil infiltration, which causes progressive airway damage through production and release of activated oxygen metabolites and proteolytic enzymes, including neutrophil elastase (Fick, Chest 96:158-164 (1989)). Moreover, excessive neutrophil response and consequent lung deterioration observed in CF patients may not be exclusively associated with P. aeuruginosa infection itself. Signs of inflammation, including neutrophil and monocyte infiltration, and high levels of pro-inflammatory cytokines have been observed in children experiencing pulmonary inflammation in the absence of infection and in bronchoalveolar lavage (BAL) fluid of infants with CF prior to a detectable P. aeuruginosa infection (Khan et al., Am J Respir Crit. Care Med. 151:1075-82 (1995)). Thus, it is possible that inflammation in the CF lung may occur independently of infection, or that even minor infection may induce an unusually robust and persistent inflammatory reaction. Lung inflammation, and the associated deterioration of bronchoaveolar tissue, may begin quite early in life and persist indefinitely, either in the context of an infection-independent inflammatory mechanism, or in the context of a perpetuated immune response to an infection that is never fully resolved.

Strategies designed to decrease neutrophil influx into the lung and prevent neutrophil activation could decrease inflammation associated with perpetual or excessive neutrophil broncheolar influx and diminish chronic inflammation and associated tissue injury. Therapies that attenuate inflammatory response, both through the use of corticosteroids and high-dose regimens of non-steroidal anti-inflammatory drugs (NSAIDS), represent a promising strategy for treatment, but are limited by the occurrence of adverse side effects with the long-term use.

Two altered states of P. aeuruginosa growth are also thought to play a role in the pathogenesis and persistence of infection in CF patients: mucoid conversion and formation of biofilms. Mucoid conversion occurs in vivo and is associated with establishment of chronic infection. Initial colonization of CF lungs by P. aeuruginosa can be eradicated by antibiotic therapy. However, in studies of sputum sample isolates at later times, a correlation has been observed between the appearance of colony morphology associated with conversion to mucoid growth and an inability to clear the infection, even with aggressive antibiotic treatment (Frederiksen et al., Pediatr. Pulmonol. 23: 330-335 (1997)). The mucoid phenotype is characterized by overproduction of alginate, a capsule-like polysaccharide that is thought to affect bacterial adherence, manifest resistance to neutrophil infiltration, neutralize oxygen radicals, serve as a barrier to phagocytosis, and constitute a barrier that renders mucoid bacteria refractory to antibiotic therapy (Evans and Linker, J. Bacteriol. 116: 915-924 1993; Govan and Deretic, Microbiol. Rev. 60: 539-574 (1996)). The mucoid phenotype is often due to mutations that truncate the mucA gene product, an anti-sigma factor that normally negatively regulates the operon encoding alginate (Martin et al., Proc. Natl. Acad. Sci. USA 90: 8377-8381 (1993)). mucA mutations are found in 84% of mucoid P. aeuruginosa isolates (Boucher et al., Infect. Immun. 65:3838-46 (1997)), an observation that may account for the high incidence and persistence of mutator strains in CF patients (Oliver et al., Science 288:1251-1253 (2000)).

Interestingly, one source of selection for mucoid conversion may be the presence of oxygen radicals produced by infiltrating neutrophils. Indeed, such radicals may play a causal role in the conversion of non-mucoid cells to the mucoid phenotype: exposure of P. aeuruginosa to oxygen radicals in vitro leads to mucoid conversion (Mathee et al., Microbiology 145: 1349-1357 (1999)). Thus, the exaggerated inflammatory immune response mounted by the CF patient in the infected lung may play an additional role in the CF disease process by influencing the emergence of intractable forms of P. aeuruginosa. This, in turn, suggests that selective attenuation of neutrophil infiltration and/or activation might prevent or attenuate mucoid conversion, thereby perpetuating the efficacy of antibiotic therapies and delay or prevent conversion of non-mucoid P. aeuruginosa to the more virulent mucoid form.

One desirable ultimate goal of an effective therapy for inflammation in the CF lung would be the attenuation of neutrophil elastase release and oxide radical production without profoundly diminishing other neutrophil functions or the responsiveness of other components of the immune response, thereby maintaining the ability to effectively combat the bacterial infection while minimizing inflammation-related tissue damage in the lung. As mentioned above, neutrophil chemotaxis and activation is mediated by several small signaling molecules that form extracellular gradients. However, formylated peptides differ from other less potent signaling molecules in that they elaborate a robust and complete neutrophil response. In contrast, other effectors, such as C5a and leukotrienes, potently stimulate neutrophil chemotaxis and antigenic body engulfment, but do not effectively induce degranulation and its accompanying tissue degenerative effects of elastase and superoxide production (Klinker et al., Biochem. Pharmacol. 48: 1857-1864 (1994)).

Formyl peptide receptors (FPRs) are G protein coupled receptors expressed primarily in neutrophils and some cells of macrophage or phagocyte lineage. The most potent and best-characterized ligands for these receptors are peptides or protein fragments containing N-formyl methionine residues, a hallmark of proteins of prokaryotic origin. As such, these peptides serve as potent immunological homing signals for sites of bacterial infection, signaling several phases of neutrophil response and activation, including chemoattraction, stimulation of production and release of immunosignaling molecules (e.g., interleukins, cytokines, etc.), as well as degranulation, a cellular process that includes the production and release of both chemical (e.g., hydrogen peroxide and other reactive oxygen radical species) and enzymatic agents (e.g., elastase and other digestive enzymes) capable of mediating destruction of the foreign agent or pathogen.

In humans, three related FPR family members have been identified: the eponymous formyl peptide receptor (FPR), as well as two other receptors, FPRL1 and FPRL2. FPRL1 and FPRL2 are related to FPR by sequence homology but appear to be functionally distinct. Lipoxin A₄ and serum amyloin A (SAA) have been proposed as natural ligands for FPRL1R (Takano et al., J. Exp. Med. 185:1693-1704 (1997)). Interestingly, the mouse homolog (muFPR), which is 70% identical to the human receptor, binds formyl-methionine-leucine-phenylalanine (fMLF) with approximately 200 fold lower in affinity than the human receptor, and elicits a proportionately weaker intracellular response in mouse cells (Gao and Murphy, J. Biol. Chem. 268: 25395-15401 (1993)). Moreover, fMLF is a relatively poor chemoattractant for rodent neutrophils and phagocytes (Sasagawa et al., Immunol. Phamacol. Immunotoxicol. 14: 625-635 (1992); Walker et al., J. Leuk. Biol. 50: 600-606 (1991), suggesting that this particular response may play a qualitatively or quantitatively different role in orchestration of immune response in rodents than in humans.

Although fMLF mediated chemoattraction is among the best known and characterized immunomodulatory signaling systems, little is known about the in vivo contribution of fMLP signaling relative to other chemoattractants, including various components of the complement system and the many known chemokines, under physiological conditions. This is especially true of roles that fMLF signaling might play in human diseases and disorders, including any etiologic roles in acute or chronic inflammation. Although inflammation is a critical event in localized immune response, serving to initiate and amplify signaling cascades that congregate and modulate cells and cellular activities critical for clearing of an infection, loss of control of this signalling process could potentially lead to any number of pathological inflammatory states or associated conditions.

The potential benefits of mouse models of human disease are well known and have been widely useful in both basic scientific discovery and drug discovery. A primary requirement for such models is that they reflect analogous relevant features of the biological process or disease. Dissecting the relative roles of various chemoattractants in immune response and inflammation can be done using transgenic or knockout mice. However, it is now clear that many aspects of the mouse immune response are quite distinct from those of other mammals, primates, and humans.

FPR knockout mice have been generated and studied (Gao et al., J. Exp. Med. 189:657-662 (1999)). Although these mice exhibit increased mortality in response to intravenous Listeria monocytogenes challenge, these mice are not susceptible to spontaneous infections, and display neither a general compromise of immunity nor other developmental or physiological abnormalities. However, in light of the fact that fMLF is such a poor ligand and signaling molecule for mouse cells (i.e. cells expressing the mouse receptor), the muFPR knockout mouse does not accurately model the impact of loss or attenuation of FPR response in humans. As a corollary to this, it is clear that although a rational circumstantial case can be made for the role of FPR signalling in pathological processes or diseases in humans, the lack of neutrophil response to N-formyl peptides in normal mice or rodents necessarily implies that systematic experimental cause evidence of such involvement has not been forthcoming through the use of conventional rodent models for disease, or inflammation-associated pathological conditions and disorders. Indeed, these facts lead to the conclusion that a mouse model truly suitable for the examination of FPR response and for the study of its role in human immune response and disease does not presently exist.

### SUMMARY OF THE INVENTION

In one aspect, this invention features a transgenic mouse, the genome of which contains a polynucleotide encoding a human formyl peptide receptor (hFPR). In one embodiment, the polynucleotide is operably linked to an expression control sequence that includes a promotor, desirably the CD11b promotor, capable of expressing a DNA sequence that is substantially identical to that of SEQ. ID NO. 1. In an additional embodiment, the hFPR is encoded by a polynucleotide that hybridizes under high stringency conditions to the coding sequence of hFPR. Desirably, the expression is controlled such that the hFPR is expressed in leukocytes. Most desirably, the hFPR-expressing white blood cell is a neutrophil or a macrophage.

In another embodiment, the mouse can be a CD-1® Nude mouse, a CD-1 mouse, a NU/NU mouse, a BALB/C Nude mouse, a BALB/C mouse, a NIH-III mouse, a SCID™ mouse, an outbred SCID™ mouse, a SCID Beige mouse, a C3H mouse, a C57BL/6 mouse, a DBA/2 mouse, a FVB mouse, a CB17 mouse, a 129 mouse, a SJL mouse, a B6C3F1 mouse, a BDF1 mouse, a CDF1 mouse, a CB6F1 mouse, a CF-1 mouse, a Swiss Webster mouse, a SKH1 mouse, a PGP mouse, or a B6SJL mouse. In yet another embodiment, the transgenic mouse is female.

In a preferred embodiment, the transgenic mouse whose genome contains a polynucleotide encoding an hFPR further contains a homozygous deletion or disruption of the cystic fibrosis transmembrane conductance regulator (CFTR) gene. Thus, the mouse expresses the hFPR but not the mCFTR gene products.

Another aspect of the invention features a cell line derived from a transgenic mouse of the invention.

In yet another aspect, the invention features a method for producing a transgenic mouse by providing an exogenous expression vector that contains a nucleotide sequence of the CD11b promotor operably linked to a nucleotide sequence encoding the human formyl peptide receptor. The expression vector is introduced into a fertilized mouse oocyte, which is then allowed to develop to term. The desired mouse is characterized by expression of hFPR. In preferred embodiments, the mouse has an increased response to one or more appropriate neutrophil activating stimuli (e.g. N-formyl peptides or other FPR agonists), and observation of the effects such stimuli, relative to the responses of comparable non-transgenic animals.

The invention also features a method for measuring an inflammatory response having the steps of: providing a transgenic mouse of the invention, physiologically stressing the mouse sufficient to cause an increased neutrophil recruitment to the area of physiological stress, obtaining a blood or tissue sample from the mouse, and measuring neutrophil response, neutrophil infiltration, neutrophil degranulation, or any combination thereof. The physiological stress can be caused by chemical agents that include, for example, formylated peptides, desirably fMLP, antigenic protein fragments, agonists of the human formyl peptide receptor, prokaryotic cells, prokaryotic cell lysates, or lysates from eukaryotic cells containing mitochondrially derived N-formyl peptides.

In one embodiment, the area of physiological stress is the lung of the mouse of the invention. Desirably, the stress is acute or chronic lung inflammation. The stress can be induced by a prokaryotic organism, desirably one with pathogenic properties, such as P. aeruginosa, most desirably of a mucoid phenotype. When measuring the inflammatory response, the rate of appearance and/or disappearance of P. aeruginosa can also be assessed.

In another embodiment, the area of physiological stress is the gastrointestinal tract of the transgenic mouse and the mouse is used as a model for inflammatory bowel disease, for example, Crohn's disease. In other embodiments, the physiological stress is acute skin inflammation, peritonitis, or ischemic reperfusion injury.

In another embodiment, the physiological stress is associated with a genetic defect (e.g., homozygous deletion), such as, for example, defects seen in mammals with a cystic fibrosis genotype.

In another aspect, the invention features a method for screening or characterization of one or more compounds or pharmaceutical compositions for their ability to inhibit neutrophil recruitment using a transgenic mouse that produces in its leukocytes recombinant human formyl peptide receptor. The steps of the method include, providing a mouse of the invention, physiologically stressing the mouse sufficient to cause an increased neutrophil recruitment to the area of physiological stress and neutrophil activation, administering a compound to interact with neutrophils, and measuring neutrophil response, neutrophil infiltration, neutrophil degranulation, or any combination thereof. Effective compounds in this screen inhibit neutrophil recruitment or one or more neutrophil functions associated with activation, including but not limited to degranulation, elastase activity, or generation of reactive oxygen species.

In yet another aspect, the invention features a method for screening compounds for the prevention of neutrophil recruitment using a transgenic mouse that produces in its leukocytes recombinant human formyl peptide receptor. The steps of the method include, providing a mouse of the invention, administering the compound, physiologically stressing the mouse, and measuring neutrophil response, neutrophil infiltration, neutrophil activation, neutrophil degranulation, or any combination thereof. Effective compounds in this screen prevent neutrophil recruitment.

The physiological stress induced in either screening method can be mediated by agents such as formylated peptides (of synthetic, prokaryotic, or mitochondrial origin), antigenic protein fragments, synthetic non-peptidic agonists of the human formyl peptide receptor, prokaryotic cells, or prokaryotic cell lysates. The stress-inducing prokaryotic cell can be one with pathogenic properties and is preferably P. aeruginosa, most preferably P. aeruginosa of a mucoid phenotype.

In any of the screening methods in which the transgenic mouse is challenged by inoculation with P. aeruginosa, alternatively or in addition to measuring neutrophil activation, the conversion of P. aeruginosa from a non-mucoid into a mucoid phenotype can also be assessed. The presence of hydrogen peroxide (H₂O₂) induces the phenotypic conversion into the mucoid form. Accordingly, compounds that inhibit neutrophil activation, such as hFPR antagonists, reduce the local H₂O₂ production and slow or prevent the phenotypic conversion.

In another aspect, therapeutically effective amounts of hFPR antagonists, such as, for example, those presented in Table 1, are used to treat diseases or conditions having undesired or inappropriate neutrophil activation. Such diseases include, for example, cystic fibrosis, inflammatory bowel disease, and Crohn's disease. When treating CF, FPR antagonists may prevent or reverse the mucoid or biofilm phenotypes of P. aeruginosa. The hFPR antagonists can be administered by any appropriate route, depending upon the specific disease or condition being treated. For example, hFPR antagonists administered for the treatment of cystic fibrosis can be administered by inhalation or parenteral injection (i.m., i.v., or s.c.). Crohn's disease or inflammatory bowel disease may be treated by oral or rectal (suppository or enema) administration of FPR antagonists.

TABLE 1
hFPR Antagonists

| | Binding | | ED₅₀ | ID₅₀ | |
|---|---|---|---|---|---|
| FPR Ligands | (K_{d}) | | (Agonist) | (Antagonist) | |
| formyl-Met-Leu-Phe | 0.02 | μM | 0.02 μM | | |
| formyl-Met-Phe-Phe | ND | | 0.008 μM | | |
| CsH | 0.1 | μM | | 0.24 | μM |
| Boc-Phe-Leu-Phe-Leu-Phe | 1.46 | μM | | 1.04 | μM |
| phenyl-Met-Leu-Phe | 0.03 | μM | | 0.04 | μM |
| 4-chlorophenyl-Met-Leu-Phe | 0.002 | μM | | 0.001 | μM |
| 4-methoxyphenyl-Met-Leu-Phe | 0.002 | μM | | 0.001 | μM |
| H-Phe-D-Leu-Phe-D-Leu-Phe | 0.8 | μM | | 0.1 | μM |
| isopropyl-Phe-D-Leu-Phe-D- | 0.2 | μM | | 0.5 | |
| Leu-Phe | | | | | |
| phenyl-Phe-D-Leu-Phe-D-Leu-Phe | 0.05 | μM | | 0.5 | μM |
| 1-adamantyl-Phe-D-Leu-Phe-D- | 0.02 | μM | | 0.3 | μM |
| Leu-Phe | | | | | |
| m-tolyl-Phe-D-Leu-Phe-D-Leu-Phe | 0.2 | μM | | 0.02 | μM |
| H-Leu-Asp-Leu-Leu-Phe-Leu | ND | | | 2.0 | μM |
| i-Boc-Met-Leu-Phe | 0.57 | μM | | 0.25 | μM |
| Cbz-Met-Leu-Phe | 2.7 | μM | | 0.42 | μM |

By “neutrophil activation” (and quantitative measurement or qualitative assessment thereof) is meant an indicator of neutrophil response, including, for example, neutrophil infiltration, neutrophil degranulation, neutrophil rolling, neutrophil chemotaxis, increased expression or activity of various catabolic or degradative enzymes (e.g., elastases), oxidative burst, production or release of hydrogen peroxide and other highly reactive oxygen species, intracellular calcium flux, cell polarization, and changes in inositol metabolism and signaling. Other determinants of neutrophil activation include increased expression and production of leukotrienes, complement, chemokines, cytokines, chemoattractant factors, interleukins, or interferons. Methods for measuring these are also well known to those skilled in the art. (See e.g., William E. Paul, Fundamental Immunology, Lippincott Williams and Wincing Publishers. 1999; John E. Coligen et al., Current Protocols in Immunology, John Wiley & Sons, New York, N.Y., 1999.)

By “symptoms of CF” is meant any qualitative or quantitative change in the mouse with respect to any aspect of the pathology of CF observed in humans suffering from the disease. These include, but are not limited to the following changes in CF mice expressing hFPR, when compared to CF mice if equivalent genetic background that do not express the hFPR transgene: susceptibility to bacterial infections of the lung (naturally occurring or experimentally induced), inability to resolve a bronchial infection, occurrence of bacterial infection that is refractory to antibiotic treatment, the presence or appearance of mucoid or biofilm forms of P. aeruginosa, increased neutrophil infiltration in the lung or brochoaveolar fluids, increased neutrophil infiltration or activation in tissues of the lung or brochoaveolar fluids, increased localized expression or production of chemokines, cytokines, chemotactic factors, chemoattracts, or interleukins by neutrophils, degradation of lung tissues.

By “leukocyte” is meant a white blood cell. Leukocytes can be neutrophils, macrophages, or lymphocytes, such as T-cells, B-cells, or other blood cells of the immune system.

By “pharmaceutical composition” is meant any composition which contains at least one therapeutically or biologically active agent and is suitable for administration to a patient. For the purposes of this invention, pharmaceutical compositions include, but are not limited to, oral tablets and solutions, topical creams, lotions, and gels, inhalants, injectables suitable for intravenous, intramuscular, or subcutaneous administration, suppositories, and enemas. Any of these formulations can be prepared by well known and accepted methods of art. See, for example, in Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York.

By “therapeutically effective amount” is meant an amount sufficient to provide medical benefit. When administering hFPR antagonists to a human patient according to the methods described herein, a therapeutically effective amount is usually about 1-2500 mg per dose. Preferably, the patient receives 10 mg, 100 mg, 500 mg, 750 mg, 1000 mg, 1500 mg, or 2000 mg of the hFPR antagonist in each dose. Dosing is typically performed 1-5 times each day.

By “operably linked” is meant that a nucleic acid molecule and one or more regulatory sequences (e.g., a promoter) are connected in such a way as to permit expression and/or secretion of the product (i.e., a polypeptide) of the nucleic acid molecule when the appropriate molecules (e.g., transcriptional activator proteins) are bound to the regulatory sequences.

By “substantially identical” is meant a nucleic acid exhibiting at least 75%, but preferably 85%, more preferably 90%, most preferably 95%, or even 99% identity to a reference amino acid or nucleic acid sequence. The length of comparison sequences will generally be at least 60 nucleotides, preferably at least 90 nucleotides, and more preferably at least 120 nucleotides.

By “transgenic” is meant any cell which includes a nucleic acid sequence that has been inserted by artifice into a cell, or an ancestor thereof, and becomes part of the genome of the animal which develops from that cell. Preferably, the transgenic animals are transgenic mammals (e.g., rodents or ruminants). Preferably the nucleic acid (transgene) is inserted by artifice into the nuclear genome.

A “transgenic animal” refers to any animal, preferably a non-human mammal, bird or an amphibian, in which one or more of the cells of the animal contain heterologous nucleic acid introduced by way of human intervention, such as transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within the chromosome, or it may be extra-chromosomally replicating DNA. In the typical transgenic animals described herein, the transgene causes cells to express a recombinant form of one of the subject polypeptide, e.g. either agonistic or antagonistic forms. However, transgenic animals in which the recombinant gene is silent are also contemplated, as for example, the FLP or CRE recombinase dependent constructs described below. Moreover, “transgenic animal” also includes those recombinant animals in which gene disruption of one or more genes is caused by human intervention, including both recombinant and antisense techniques.

By “high stringency conditions” is meant any set of conditions that are characterized by high temperature and low ionic strength and allow hybridization comparable with those resulting from the use of a DNA probe of at least 40 nucleotides in length, in a buffer containing 0.5 M NaHPO₄, pH 7.2, 7% SDS, 1 mM EDTA, and 1% BSA (Fraction V), at a temperature of 65 C, or a buffer containing 48% formamide, 4.8×SSC, 0.2 M Tris-Cl, pH 7.6, 1×Denhardt's solution, 10% dextran sulfate, and 0.1% SDS, at a temperature of 42 C. Other conditions for high stringency hybridization, such as for PCR, Northern, Southern, or in situ hybridization, DNA sequencing, etc., are well-known by those skilled in the art of molecular biology. See, e.g., F. Ausubel et al., Current Protocols in Molecular Biology, John Wiley & Sons, New York, N.Y., 1998, hereby incorporated by reference.

By “vector” is meant a DNA molecule, usually derived from a plasmid or bacteriophage, into which fragments of DNA may be inserted or cloned. A vector will contain one or more unique restriction sites, and may be capable of autonomous replication in a defined host or vehicle organism such that the cloned sequence is reproducible. A vector contains a promoter operably linked to a gene or coding region such that, upon transfection into a recipient cell, an RNA is expressed.

### BRIEF DESCRIPTION OF THE DRAWINGS

FIG. 1 is a schematic diagram of the plasmid from which the DNA fragment for generation of the hFPR transgenic mouse was derived. This plasmid contains the CD11 promoter, followed by an intron, coding sequences for the hFPR polypeptide, followed by a polyadenylation signal. A DNA fragment containing these elements is excised using the Nod and KpnI sites; this isolated fragment is then used to generate the transgenic animal vector encoding the hFPR polypeptide under the control of the CD11b promoter. This vector was used to create the hFPR-expressing transgenic mice of this invention.

FIG. 2 is the nucleotide sequence of a DNA Fragment encoding the human formyl peptide receptor (hFPR). The sequence contains the hFPR promoter and intronic sequences.

FIG. 3 is the nucleotide sequence of a DNA Fragment encoding the human formyl peptide receptor (hFPR), referred to as SEQ. ID NO. 1.

### DETAILED DESCRIPTION OF THE INVENTION

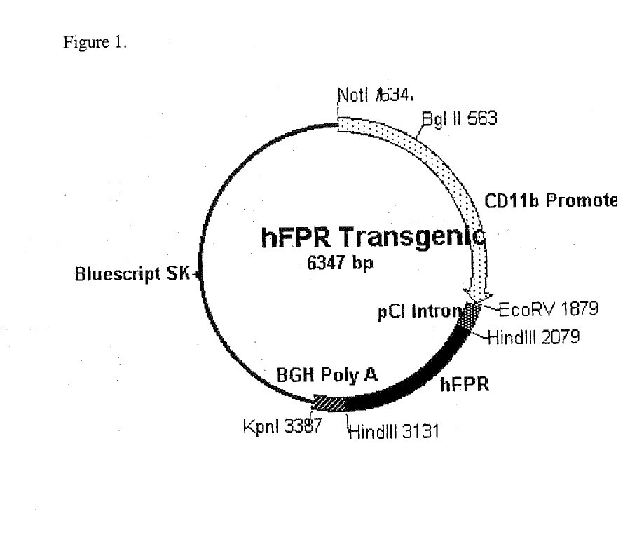

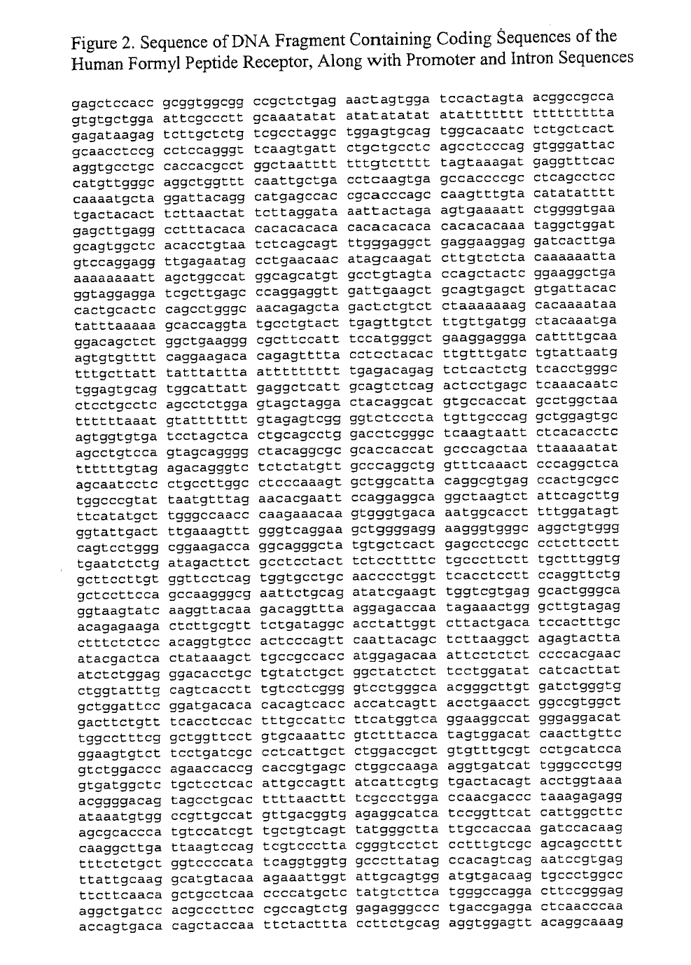

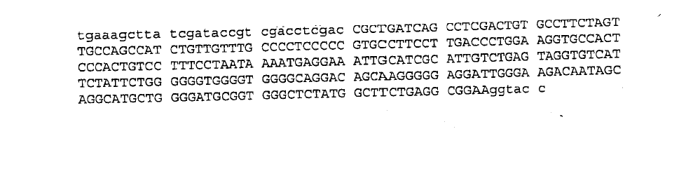

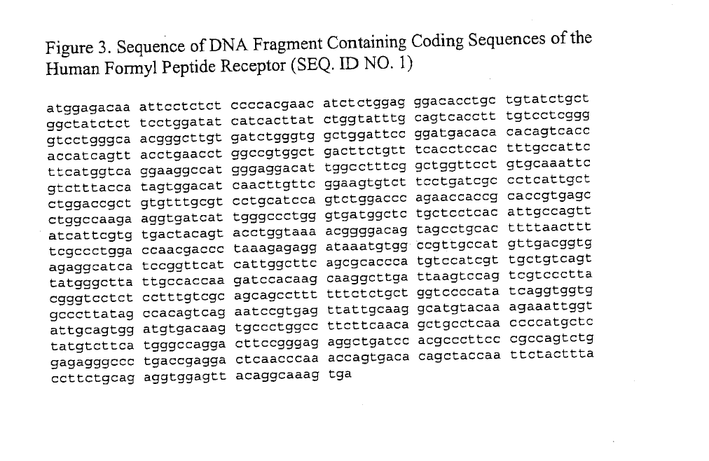

One aspect of this invention describes mice that ectopically express the human formyl peptide receptor (hFPR), with preference for mice in which expression is focused within tissue or cell types generally consistent with its function in immunity. Specifically, in which expression occurs within and is generally restricted to neutrophils and macrophages. In one specific embodiment, as shown in FIG. 1, hFPR expression is sponsored by the CD11b promoter, which is known to be active according to such a profile. Although the mouse promoter is shown in this example, other appropriate promoters are contemplated, including analogous transcriptional control regions of humans other mammals, as well as formyl peptide promoters themselves. In the plasmid construct used to generate the transgenic mouse in this example, an intron is included within the actively transcribed region of the plasmid to support stable expression and efficient processing of the resulting transcript. Similarly, the construct also contains typical transcript termination/polyadenylation signals.

The polynucleotides encoding hFPR can be inserted into the genome of any strain of mouse known in the art including, for example, CD-1® Nude mice, CD-1 mice, NU/NU mice, BALB/C Nude mice, BALB/C mice, mice, SCID™ mice, outbred SCID™ mice, SCID Beige mice, C3H mice, C57BL/6 mice, DBA/2 mice, FVB mice, CB17 mice, 129 mice, SJL mice, B6C3F1 mice, BDF1 mice, CDF1 mice, CB6F1 mice, CF-1 mice, Swiss Webster mice, SKH1 mice, PGP mice, and B6SJL mice. In addition, other genetic alterations can be made to the transgenic mouse of the invention, desirably including those that facilitate the study of inflammatory diseases. A highly desirable additional mutation is the homozygous deletion of the gene allele that is responsible for cystic fibrosis.

The transgenic mouse of this invention can be used experimentally to examine the role of the hFPR in neutrophil inflammation, in which the mouse can be challenged by a variety of stimuli, and comparing neutrophil activation or function in these transgenic animals to equivalent non-transgenic animals of analogous genetic background. Moreover, such mice can be used to determine the role of hFPR activation in the etiology or pathology of particular diseases or disorders. For example, the transgenic mouse of this convention can be crossed with CF mice, resulting in lines of CF mice that stably express hFPR. These mice can be examined for symptoms of CF, and the presence or severity of such symptoms can be compared to non-transgenic CF mice of equivalent genetic background. The role of hFPR in the etiology or pathology of IBD or Crohn's disease can be established using the transgenic mouse of this invention by challenging these mice through standard protocols known in the art for inducing symptoms of these diseases in mice and other animal models. The timing of onset, severity, and resolution of such symptoms in these mice can be compared to that of non-transgenic mice of analogous or equivalent genetic background.

The mouse of the invention can also be used to examine various aspects of neutrophil response upon direct introduction of formylated peptides (of synthetic, prokaryotic, or mitchondrial origin), or fragments of proteins containing N-terminal formylated peptides, as well as to challenge by procaryotic organisms or pathogens.

N-formyl peptides are important signals for stimulation of the immune response to bacterial infection. However, such peptides are also found in the mitochondria of eukarotic cells. Thus, such peptides may also serve as signals for neutrophil recruitment and activation in tissues undergoing cell death, necrosis, or apoptosis. The role of such peptides of mitochondrial origin as stimulators of neutrophil response, neutrophil-mediated inflammation, or neutrophil-mediated tissue damage has not been studied in large part due to the lack of a facile and suitable animal model for the controlled introduction or induction of such peptides. One element of this invention describes the use of hFPR transgenic mice to study the role of N-formyl peptides of mitchondrial origin in inflammation, inflammatory disorders, diseases, or other pathological conditions associated with signaling by such peptides. Accordingly, this invention also contemplates the therapeutic use of FPR antagonists in the treatment or amelioration of conditions or symptoms derived from manifestations of such signaling. In these examples, neutrophil infiltration and degranulation can also be examined by methods known in the art, as those described, for example, by van Eeden, et al. in J. Immunol. Methods 232:23-43 (1999). Examples of physiological stresses that can result in a response that can be measured by the mouse of the invention include inflammation of the lung, inflammation of the gastrointestinal tract, acute skin inflammation, peritonitis, or ischemic reperfusion injury.

There are a number of tissues in which either procaryotic flora exist under normal non-pathological conditions, or in which flora are altered in association with a disease or pathological condition. For example, the colon and other components of the lower digestive tract contain endogenous flora of E. coli. and other procaryotic organisms. In this case, although the presence of these organisms brings about high levels of formyl peptides, inflammatory and immune responses to these potent signaling molecules must be sufficiently attenuated to avoid adverse or pathological inflammatory events under normal conditions. Indeed, dysregulation or loss of the attenuation of this response may lead to pathologies associated with inflammation of the colon or lower digestive tract, including inflammatory bowel disease (IBD) and Crohn's disease.

This invention also describes a transgenic mouse expressing the hFPR that may be used to study inflammatory processes of the digestive tract. In a preferred embodiment, standard experimental protocols or procedures for inducing symptoms of IBD or Crohn's disease in mice (e.g. abrasion or irritation) may be used to challenge these mice, and both qualitative well and quantitative aspects of immune and inflammatory response may be assessed. One particular aspect of such examination would include (but not be limited to) neutrophil infiltration and activation (i.e., degranulation, release of elastase and reactive oxygen radicals). Other aspects would include the timing, progression, and severity of known symptoms of IBD or Crohn's disease in these mice compared to wild type and unchallenged mice.

Use of hFPR Transgenic Mice to Study Cystic Fibrosis

The lungs of individuals with cystic fibrosis are chronically colonized by P. aeruginosa, an organism that does not pose a significant health problem for normal individuals. Profound and perpetuated neutrophil infiltration and activation are also hallmarks of the human cystic fibrotic lung. Although it is known that the cystic fibrosis gene defect causes loss-of-function of a chloride channel, and that this results in changes in the osmotic physiology of the affected lung, the relationship between this condition and the etiology of chronic bacterial infection, as well as the profound and protracted inflammation and tissue damage that characterize the disease, are not understood.

Following cloning of the CF gene and identification of the channel protein mutation responsible for cystic fibrosis (CF), the homologous a mouse model was developed in which the mouse homolog was knocked out (Snouwaert et al., J. Clin. Invest. 11:2810-2815 (1992); Clarke et al., Science 257:1125-1128 (1992)). Although these mice weigh less than normal controls and have a slightly increased mortality, as well as digestive and reproductive defects, they do not develop frank symptoms of CF disease (van Heeckeren et al., J. Clin. Invest. 11:2810-2815 (1997)). Utility of these mice has been primarily in the study of alterations in lung physiology associated with CF. However, since these mice display none of the inflammatory features of CF, and are not susceptible to colonization and chronic infection by P. aeruginosa, contribution of this model to these aspects of CF etiology, disease, pathology or symptoms has been minimal.

These intrinsic differences between the CF mouse and pathological hallmarks and features of the human disease point out potentially important deficiencies in use of this mouse as a complete surrogate model for study of the human disease. Many or all of these differences are directly and indirectly associated with inflammatory processes in the rodent versus other animals (and include aspects of neutrophil response, e.g., the documented lack of responsiveness of mouse compared to human neutrophils and FPRs to formylated peptide ligands). Indeed, lack of formyl peptide receptor responsiveness by the mouse neutrophil could account for significant differences in the physiological function of mouse neutrophils compared to those of human origin. For this reason, any disease, inflammatory disorder in humans that is causally linked to formyl peptide-mediated neutrophil signaling would not be manifest in a normal mouse or rodent. The transgenic mouse of this invention provides a means of determining the physiological role of hFPR signaling in a mouse model and importantly, allows systematic comparison of the hFPR transgenic mouse to a normal mouse in which this signalling is absent. Accordingly, use of this mouse will for the first time facilitate elucidation of the role of such formyl peptide signaling in the etiology or pathology of human diseases or inflammatory disorders using a mouse model.

Another transgenic mouse of this invention expresses the mFPR in a CF mouse genetic background (e.g., CFTR deletion). Expression of hFPR in the neutrophils of CF mice allows examination of the role of formyl peptides in the neutrophil-associated pathologies and symptoms of CF (CF symptoms that are well documented in humans but not observed in existing CF mouse models). It is possible that inflammation reminiscent of human CF will be observed in CF mice upon expression of hFPR. As mentioned above, many such symptoms are not a feature of existing CF mouse models. Such an observation would certainly lend credence to the hypothesis that fMLP response plays an important role in modulating the intense and prolonged inflammatory response and tissue damage in the CF lung and the severity of its consequences.

Such considerations take on additional importance in light of the observation that physiological conditions associated with neutrophil response may play a pivotal role in determining the growth state of P. aeruginosa. Two altered growth states of P. aeuruginosa are also thought to play a role in the pathogenesis and persistence of infection in CF patients: mucoid conversion and formation of biofilms. Mucoid conversion occurs in vivo and is associated with establishment of chronic infection. This phenotype is characterized by overproduction of alginate, a capsule-like polysaccharide that is thought to affect bacterial adherence, mediate resistance to neutrophil infiltration, neutralize oxygen radicals, serve as a barrier to phagocytosis, and constitute a barrier that is refractory to antibiotic therapy (Evans and Linker, J. Bacterial. 116:915-924 (1993); Govan and Deretic, Microbial. Rev. 60:539-574 (1996)). The mucoid phenotype is often due to mutations that truncate the mucA gene product, an anti-sigma factor that normally negatively regulates the operon encoding alginate (Martin et al., Proc. Natl. Acad. Sci. 90:8377-8381 (1993)). mucA mutations are found in 84% of mucoid P. aeuruginosa isolates (Boucher et al., Infect. Immun. 65:3838-46 (1997)), an observation that may be accounted for by the high incidence and persistence of mutator strains in CF patients (Oliver et al., Science 288:1251-1253 (2000)). Initial colonization of CF lungs by P. aeuruginosa can be eradicated by antibiotic therapy. However, in studies of sputum sample isolates at later times, a correlation has been observed between the appearance of colony morphology associated with conversion to mucoid growth and an inability to clear the infection, even with aggressive antibiotic treatment (Frederiksen et al., Pediatr. Pulmonol. 23: 330-335 (1997)).

Even transient in vitro exposure of P. aeuruginosa to concentrations of oxygen radicals that are readily achieved physiologically during robust neutrophil response leads to mucoid conversion (Mathee et al., Microbiology 145:1349-1357 (1999)). Therefore, it is possible that infiltrating neutrophils may contribute significantly to mucoid conversion. Thus, the exaggerated and prolonged neutrophil inflammatory immune response mounted by the CF patient in the infected lung may contribute directly to the emergence of intractable forms of P. aeuruginosa, and suggests that selective attenuation of neutrophil infiltration and/or activation might perpetuate the efficacy of antibiotic therapies and delay or prevent conversion of non-mucoid P. aeuruginosa to the more virulent mucoid form. Indeed, the presence of bacteria, along with lysed cells and cell debris, as well as continued residence of viable and mucoid bacteria, may constitute a “vicious cycle” of perpetual neutrophil provocation, immune response, and emergence of refractory bacterial populations. In such a scenario, it may be extremely beneficial to break this cycle in a manner that decreases the intensity and nature of neutrophil response without compromising immune response.

A transgenic mouse expressing the hFPR in a CF background may be used to determine whether clearance of lung infection by P. aeruginoisa infection is altered in the context of robust formyl peptide response by mouse neutrophils. These mice can be used to measure whether human FPR alters the response of the CF lung to this challenge, both with respect to effectiveness in clearing the infection, and the extent to which neutrophil infiltration, activation, and inflammation occurs either in the presence or absence of exogenous P. aeruginoisa infection. Timing and quantitation of mucoid conversion may also be assessed. Suitable transgenic mice may be created by crossing transgenic mice expressing hFPR with CF mice.

Identification of Therapeutic hFPR Antagonists

Antagonists of the hFPR, suitable for human therapeutic use for the treatment of inflammation diseases associated with the formyl peptide receptor. Specifically, altered inflammatory response in the cystic fibrotic lung or in models of IBD or Crohn's disease can be induced in these mice and the effectiveness of hFPR antagonists to reverse or ameliorate the symptoms, progression, severity, or other features associated with the induced pathology or disease or disorder. Cyclosporin H is an example of such an antagonist.

For example, the hFPR transgenic mouse of this invention, in which symptoms of IBD or Crohn's disease have been experimentally induced, can be treated with various doses of CsH or other hFPR antagonists, and the ability of such treatment to attenuate such symptoms or shorten their duration can be assessed. Using the CF mouse expressing hFPR can be examined for symptoms of CF in the presence or absence of treatment by CsH or other hFPR antagonists, and the severity or duration of these symptoms can be examined in these animals versus non-treated animals.

hFRP Transgenic Mouse Preparation

A DNA fragment encoding a human formyl peptide receptor polypeptide can be integrated into the genome of the transgenic mouse by any standard method well known to those skilled in the art. Any of a variety of techniques known in the art can be used to introduce the transgene into animals to produce the founder lines of transgenic animals (see, for example, Hogan et al., Manipulating the Mouse Embryo: A Laboratory Manual Cold Spring Harbor Laboratory (1986); Hogan et al., Manipulating the Mouse Embryo: A Laboratory Manual, second ed., Cold Spring Harbor Laboratory (1994), and U.S. Pat. Nos. 5,602,299; 5,175,384; 6,066,778; and 6,037,521). Such techniques include, but are not limited to, pronuclear microinjection (U.S. Pat. No. 4,873,191); retrovirus mediated gene transfer into germ lines (Van der Putten et al., Proc. Natl. Acad. Sci. USA 82:6148-6152 (1985)); gene targeting in embryonic stem cells (Thompson et al., Cell 56:313-321 (1989)); electroporation of embryos (Lo, Mol. Cell. Biol. 3:1803-1814 (1983)); and sperm-mediated gene transfer (Lavitrano et al., Cell 57:717-723 (1989)).

For example, embryonal cells at various developmental stages can be used to introduce transgenes for the production of transgenic animals. Different methods are used depending on the stage of development of the embryonal cell. The zygote is a good target for micro-injection, and methods of microinjecting zygotes are well known to (see U.S. Pat. No. 4,873,191). In the mouse, the male pronucleus reaches the size of approximately 20 micrometers in diameter which allows reproducible injection of 1-2 picoliters (pl) of DNA solution. The use of zygotes as a target for gene transfer has a major advantage in that in most cases the injected DNA will be incorporated into the host genome before the first cleavage (Brinster, et al., Proc. Natl. Acad. Sci. USA 82:4438-4442 (1985)). As a consequence, all cells of the transgenic non-human animal will carry the incorporated transgene. This will in general also be reflected in the efficient transmission of the transgene to offspring of the founder since 50% of the germ cells will harbor the transgene. Micro-injection of hFPR nucleic acid fragments were microinjected into pronuclei to generate a hFPR transgenic mouse.

The transgenic animals of the present invention can also be generated by introduction of the targeting vectors into embryonal stem (ES) cells. ES cells are obtained by culturing pre-implantation embryos in vitro under appropriate conditions (Evans et al., Nature 292:154-156 (1981); Bradley et al., Nature 309:255-258 (1984); Gossler et al., Proc. Natl. Acad. Sci. USA 83:9065-9069 (1986); and Robertson et al., Nature 322:445-448 (1986)). Transgenes can be efficiently introduced into the ES cells by DNA transfection using a variety of methods known to the art including electroporation, calcium phosphate co-precipitation, protoplast or spheroplast fusion, lipofection and DEAE-dextran-mediated transfection. Transgenes can also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (reviewed in Jaenisch, Science 240:1468-1474 (1988)). Prior to the introduction of transfected ES cells into the blastocoel, the transfected ES cells can be subjected to various selection protocols to enrich for ES cells that have integrated the transgene if the transgene provides a means for such selection. Alternatively, PCR can be used to screen for ES cells that have integrated the transgene. This technique obviates the need for growth of the transfected ES cells under appropriate selective conditions prior to transfer into the blastocoel.

In addition, retroviral infection can also be used to introduce transgenes into a non-human animal. The developing non-human embryo can be cultured in vitro to the blastocyst stage. During this time, the blastomeres can be targets for retroviral infection (Janenich, Proc. Natl. Acad. Sci. USA 73:1260-1264 (1976)). Efficient infection of the blastomeres is obtained by enzymatic treatment to remove the zona pellucida (Hogan et al., supra, 1986). The viral vector system used to introduce the transgene is typically a replication-defective retrovirus carrying the transgene (Jahner et al., Proc. Natl. Acad. Sci. USA 82:6927-6931 (1985); Van der Putten, et al. Proc. Natl. Acad. Sci. USA 82:6148-6152 (1985)). Transfection is easily and efficiently obtained by culturing the blastomeres on a monolayer of virus-producing cells (Van der Putten, supra, 1985; Stewart et al., EMBO J. 6:383-388 (1987)). Alternatively, infection can be performed at a later stage. Virus or virus-producing cells can be injected into the blastocoele (Jahner et al., Nature 298:623-628 (1982)). Most of the founders will be mosaic for the transgene since incorporation occurs only in a subset of cells which form the transgenic animal. Further, the founder can contain various retroviral insertions of the transgene at different positions in the genome, which generally will segregate in the offspring. In addition, it is also possible to introduce transgenes into the germline by intrauterine retroviral infection of the midgestation embryo (Jahner et al., supra, 1982). Additional means of using retroviruses or retroviral vectors to create transgenic animals known to the art involves the micro-injection of retroviral particles or mitomycin C-treated cells producing retrovirus into the perivitelline space of fertilized eggs or early embryos (WO 90/08832 (1990); Haskell and Bowen, Mol. Reprod. Dev. 40:386 (1995)).

A DNA fragment comprising a hFPR cDNA encoding a human formyl peptide receptor polypeptide can be microinjected into pronuclei of single-cell embryos in non-human mammals such as a mouse. The injected embryos are transplanted to the oviducts/uteri of pseudopregnant females and finally transgenic animals are obtained.

Once the founder animals are produced, they can be bred, inbred, outbred, or crossbred to produce colonies of the particular animal. Examples of such breeding strategies include but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic mice to produce mice homozygous for a given integration site in order to both augment expression and eliminate the need for screening of animals by DNA analysis; crossing of separate homozygous lines to produce compound heterozygous or homozygous lines; breeding animals to different inbred genetic backgrounds so as to examine effects of modifying alleles on expression of the transgene and the physiological effects of expression.

The present invention provides transgenic non-human mammals that carry the transgene in all their cells, as well as animals that carry the transgene in some, but not all their cells, that is, mosaic animals. The transgene can be integrated as a single transgene or in concatamers, for example, head-to-head tandems or head-to-tail tandems.

The transgenic animals are screened and evaluated to select those animals having a phenotype wherein hFPR is expressed on leukocytes. Initial screening can be performed using, for example, Southern blot analysis or PCR techniques to analyze animal cells to verify that integration of the transgene has taken place. The level of mRNA expression of the transgene in the cells of the transgenic animals can also be assessed using techniques which include, but are not limited to, Northern blot analysis of tissue samples obtained from the animal, in situ hybridization analysis, and reverse transcriptase-PCR (rt-PCR). The transgenic non-human mammals can be further characterized to identify those animals having a phenotype useful in methods of the invention. In particular, an inflammatory stimulus can be introduced to the transgenic non-human mammals of the invention and leukocytes can be examined for hFPR expression.

Additional genetic alterations can be introduced to the mouse of the present invention by techniques similar to those described above. For example, several strains of mice have been described that contain a mutation that causes cystic fibrosis (Zeiher, et al., J. Clin. Invest. 96:2051 (1995); Colledge, et al., Nat. Genet. 10:445 (1995); Zhou, et al., Science 266:1705 (1994)).

### Other Embodiments

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A transgenic mouse whose genome comprises a polynucleotide encoding a human formyl peptide receptor (hFPR).

2-13. (canceled)

14. A method for the measurement of an inflammatory response comprising: a) providing the transgenic mouse of claim 5; b) physiologically stressing said transgenic mouse, thereby causing increased neutrophil activation in the area of physiological stress; c) obtaining a blood or tissue sample from said mouse; and d) measuring neutrophil activation or infiltration.

15. The method of claim 14, wherein said physiological stress is to the lung of said mouse.

16. The method of claim 15, wherein said physiological stress is a chronic lung inflammation.

17. The method of claim 15, wherein said physiological stress is an acute lung inflammation.

18. The method of claim 14, wherein said physiological stress is to the gastrointestinal tract of said mouse.

19. The method of claim 18, wherein said physiological stress is an inflammation of the gastrointestinal tract.

20. The method of claim 18, wherein said method is used as a model for inflammatory bowel disease.

21. The method of claim 20, wherein said method is used as a model for Crohn's disease

22. The method of claim 14, wherein said physiological stress is an acute skin inflammation.

23. The method of claim 14, wherein said physiological stress is peritonitis.

24. The method of claim 14, wherein said physiological stress is ischemia reperfusion injury

25. The method of claim 14, wherein said physiologically stress is mediated by agents comprising formylated peptides, antigenic protein fragments, agonists of the human formyl peptide receptor, prokaryotic cells, or prokaryotic cell lysates, or eukaryotic cell lysates.

26. The method of claim 25, wherein said formylated peptide is formyl-methionine-leucine-phenylalanine.

27. The method of claim 25, wherein said prokaryotic cell lysate is lipopolysaccharide.

28. The method of claim 25, wherein said prokaryotic cell has pathogenic properties.

29. The method of claim 28, wherein said prokaryotic cell is Pseudomonas aeruginosa.

30-44. (canceled)

45. A method for treating a disease characterized as having increased neutrophil activation in a patient, said method comprising administering to said patient a pharmaceutical formulation comprising a therapeutically effective amount of a human formyl peptide receptor antagonist.

46. The method of claim 45, wherein said disease is cystic fibrosis, inflammatory bowel disease, or Crohn's disease.

47. The method of claim 45, wherein said human formyl peptide receptor antagonist is cyclosporin H.
